(19) United States
(12) Patent Application Publication
(10) Pub. No.: US 2003/0176760 A1
(43) Pub. Date: Sep. 18, 2003

(54) Physiologically compatible cardiac assist device and method

(75) Inventors: Reida Menshawe El Oakley, Singapore (SG); Hou-Sen Lim, Singapore (SG)

Correspondence Address: Vaughn W. North, THORPE, NORTH & WESTERN, L.L.P., P.O. Box 1219, Sandy, UT, 84091-1219, US

(73) Assignee: National University of Singapore

(21) Appl. No.: 10/365,706

(22) Filed: Feb. 10, 2003

Related U.S. Application Data
(60) Provisional application No. 60/356,305, filed on Feb. 11, 2002.

(51) Int. Cl. A61N 1/362

(52) U.S. Cl. 600/16

(57) ABSTRACT

A physiologically compatible cardiac assist device, also known as a ventricular assist device, has a pump which draws blood through a first catheter inserted into either the right atrium, the left atrium or the left ventricle and infuses the blood via a second catheter into the aorta during diastole. Attached to the second catheter is a balloon which is also inflated during diastole. The synchronized volume infusion and balloon inflation in this counterpulsation mode reduces afterload and is thus more physiologically compatible.

### CROSS-REFERENCE TO RELATED APPLICATION

This application claims the benefit of U.S. Provisional Application No. 60/356,305, filed Feb. 11, 2002.

### FIELD OF THE INVENTION

The present invention relates to cardiac assistance for off-pump coronary artery bypass surgery and the treatment of acute and chronic heart failure on a short- or long-term basis.

### BACKGROUND OF THE INVENTION

The following subsections describe the state of the art as it relates to coronary artery bypass, heart failure and cardiac assist devices that are used to conduct coronary artery bypass and to treat heart failure.

Coronary Artery Bypass Surgery

Over a million coronary artery bypass grafts (CABG) are performed worldwide every year, using standard cardiopulmonary bypass (CPB) techniques. Conventional heart-lung machines use an oxygenator, allowing the transfer of non-oxygenated blood into the aorta after oxygenation. Such devices have been used for assisting both heart and lungs but only for short-term support. However, CPB remains traumatic for the patients because it results in disturbed circulatory regulation and multi-organ injury. In particular, it may lead to Systemic Inflammatory Response Syndrome (SIRS) characterized by increased platelet dysfunction, renal insufficiency, and pulmonary insufficiency. More recent studies have demonstrated that CABG without CPB (off-pump CABG, chat is, OPCAB) results in similar clinical outcomes to the standard CABG with minimal disturbances to the circulatory system. Until recently, OPCAB was restricted to single and double vessel diseases, but with increasing experience, the technique is being used for patients with multi-vessel CABG disease, patients with critical left main stem disease or low ejection fraction, and furthermore, for non-elective emergency cases. (The use of the technique to treat left main stem disease is discussed in Dewey T M, Magee M J, Edgerton J R, et al. Off-pump bypass grafting is safe in patients with left main coronary disease. Ann Thorac Surg September 2001; 72(3): 788-91 and in Mark Y, Massimo C, Raimondo A, et al. Off-pump coronary artery bypass for critical left main stem disease: safety, efficacy and outcome. Eur J Cardiothorac Surg 2001; 19: 239-244. The use of the technique to treat low ejection fraction is discussed in 11. Abraham R, Karamanoukian H L, Jajkowski M R, et al. Low ejection fraction is not a contraindication to off-pump coronary artery surgery. Heart Surg Forum 2001; 4(2): 141-4. Finally, the use of the technique for non-elective emergency cases is dealt with in David V, Magdi H, Richad T, Mohamed A. Outcome of non-elective coronary artery bypass grafting without cardiopulmonary bypass. Eur J Cardiothorac Surg 2001; 19: 245-248.)

Although OPCAB appears superior to standard CABG in selected patients, its wider application, especially for diabetic patients with multiple-vessel disease is limited, as obtaining exposure and stabilization of coronary arteries located on the lateral and inferior wall of the heart may be problematic. OPCAB is often associated with low systemic pressures, decreased stroke volume, concomitant higher right arterial pressure, suggesting right ventricle dysfunction when the beating heart is positioned and stabilized for grafting the circumflex. (Cx), obtuse marginal (OM), and posterior descending artery (PDA). (This is discussed in greater detail in Grundeman P F, Brost C, van Herwaarden J A, et al. Hemodynamic changes during displacement of the beating heart by the Utrecht Octopus method. Ann Thorac Surg 1997; 63: S88-S92 and Grundeman P F, Brost C, van Herwaarden J A, et al. Vertical displacement of the beating heart by the Octopus tissue stabilizer: influence on coronary flow. Ann Thorac Surg 1998; 65: 1348-1352.) Although the decrease is usually temporary, it can adversely affect the coronary, cerebral and renal circulation.

A range of surgical strategies for exposure, stabilization of the beating heart coronary arteries, like Octopus stabilizer and Vacuum Assisted Heart Manipulator have been used with encouraging results. (The Octopus technique is discussed in Erik W L, Cornelius B, Jaap R L, et al. Coronary artery bypass grafting without cardiopulmonary bypass using the octopus method: results in the first one hundred patients. J Thorac Cardiovasc Surg 1998; 116: 60-67.) Furthermore, techniques of mechanical support of the left, right and both ventricles have been proposed during off-pump coronary artery procedures to prevent hemodynamic deterioration.

Heart Failure

Failure of the heart's ability to pump blood due to ischemic heart disease, viral infection, or immunological disorders is the only cardiovascular disease from which mortality continue to rise. So far, this pandemic has no cure. Rather, the only realistic palliative therapy for this syndrome is heart transplantation. However, the continual shortage of organ donors renders transplantation an unlikely therapy for more than 90% of patients with heart failure.

One of the most potentially promising treatment options for heart failure is the use of artificial blood pumps, which are otherwise known as assist devices. These assist devices displace volume in such a way so as to bypass the failing heart. The assist device may bypass the left ventricle, right ventricle or both ventricles. Many assist devices exist, and are usually classified according to their implantation technique (e.g. left atrial to aorta versus left ventricular apex to aorta), duration of use (e.g. short term versus long term) and implantability (e.g. implantable versus non-implantable), pump design (e.g. pneumatic versus push and plate), and flow dynamics (e.g. pulsatile versus continuous flow).

Most existing assist devices function satisfactorily when they take over a large proportion or entire function of the ventricle. However, during the weaning process, the ill-timed infusion and poor control of pre-load and after-load become critical issues. The current devices cannot ensure gradual transfer of the workload from the device to the ventricle, yet preserving the physiological principles of the cardiac cycle.

The normal heart circulates blood in a pulsatile manner, relying on the ventricle to generate a pressure greater than the end-diastolic aortic pressure in order to eject. However, a recovering heart simply cannot work against an artificially high end-diastolic aortic pressure (after-load). This artificially high end-diastolic aortic pressure is created by the continuous infusion of blood by the assist device. The ideal weaning conditions should start with a slow increase in preload (volume in ventricle), with a corresponding low end-diastolic aortic pressure as the heart become stronger. These two parameters are gradually increased as the ventricle adapts to the new load while maintaining blood flow volume, Unfortunately, this is usually not the condition that exists at the point of weaning for current devices. For instance, it is well documented that the Hemopump™, further discussed in the next section, maintains a high aortic pressure at full capacity. The weaning process starts under maximum afterload conditions. (PETERZEN, B., LONN, U., BABIC, A., GRANFELDT, H., CASIMIR-AHN, H., and RUTBERG, H. (1996): ‘Postoperative management of patients with Hemopump support after coronary artery bypass grafting’, Ann. Thorac, Surg., 62, pp. 495-500).

Assist Devices for OPCAB

Lönn and colleagues from the Linköping Heart Center, University Hospital in Linköping, Sweden, examined the effectiveness of OPCAB with support from a cardiac assist-axial flow pump known as the Hemopump™ (Medtronic, Minneapolis, Minn.). In their study, Lynn et al. relied upon median sternotomy for direct to the ascending aorta for the insertion of the pump into the patient's left ventricle. Upon insertion, the pump was utilized to circulate blood from the left ventricle to the ascending aorta. In addition to the well-known hazards of aortotomy and grafting of ascending aorta, this procedure allowed only the support of the left ventricle during OPCABG of single or easily accessible vessels. (This is described in greater detail in Lonn U, Bengt P, Bo Carnstam, Henrik C A, Beating heart coronary surgery supported by an Axial Blood Flow Pump. Ann Thorac Surg 1999; 67: 99-104 and in Marck M J, Acuff T E, Casimir-Ahn H, et al., Video-assisted coronary bypass grafting on the beating heart Ann Thorac Surg 1997; 63: S100-S103.)

Another study compared right and left heart bypass with the Biomedicus™ device. The study concluded that only right heart bypass could restore cardiac function during an off-pump coronary artery procedure. (This is described more fully in Grundeman P F, Brost C, Verlaan, et al. Exposure of circumflex branches in the tilted, beating porcine heart: echocardiographic evidence of right ventricular deformation and the effect of left heart bypass. J Thorac Cardiovasc Surg 1999; 18: 316-323.)

Several other devices have been used for right ventricle support. Mathison and colleagues used the right-heart support system (A-Med™ system, West Sacramento, Calif.) which consisted of a coaxial cannula placed through the right atrium. The tip of the cannula was positioned in the main pulmonary artery so that the blood was removed from the right atrium and returned to the pulmonary artery. (This is described more fully in Mathison M, Enio B, Adib D, et al. Coronary artery bypass without cardiopulmonary bypass, Ann Thorac Surg 2000; 70: 1083-5.)

A less invasive technique has been used by Andre and colleagues from the Department of Cardiothoracic Surgery, Cardiovascular Research Institute Maastricht, APCVADemic Hospital Maastricht, Netherlands. A device called the Enabler™ (Hemodynamic Systems Ltd., Yoqneam, Israel) consists of a 24F catheter that is placed via surgical preparation of the femoral vein, jugular vein or via the right atrium through the right ventricle into the pulmonary artery, so that the catheter inlet valve is positioned in the right atrium and the outlet valve is positioned in the pulmonary artery. (A full description of this set-up is to be found in Andre L, Gijs G G, Audrey A C, et al. Right ventricular support for off-pump coronary artery bypass grafting studied with bi-ventricular pressure loops in sheep. Eur J Cardiothorac Surg 2001; 19: 179-184.)

The Micropump™ (Impella, Aachen, Germany) has been used to support both ventricles during CABG, as demonstrated by Meyns and colleagues, from the Department of Cardiac Surgery, Gasthuisberg University Hospital, Belgium. These micropumps are based on the same principle as the Hemopump™. Although the micropumps are smaller, they have comparable power, which allows clinical application with peripheral access. The right pump is designed with a reverse flow impeller, allowing the blood to be forwarded from the right atrium into the pulmonary artery. The cannula is floppy and has a balloon at its tip, and this can be introduced like a Swan-Ganz catheter, guided on the tip pressure. The left ventricular pump with an outer diameter of 6.4 mm is designed for direct insertion into the left ventricle to pump blood from the ventricle to the aorta. (Further discussion is provided in Meyns Bart, Paul S, Takahiro N, et al. Micropumps to support the heart during CABG. Eur J Cardiothorac Surg 2000; 17: 169-174.)

However, in one study that used both the Enabler™ and Impella™ techniques in 17 patients, the cannula proved difficult to position in the pulmonary artery and there was also blood loss. Furthermore, full output was not achieved in some cases. (This is discussed at length in Andress Boening, Sandra F, Jan S, et al. Right ventricular assist devices for beating heart surgery maintain cardiac output but are a source for adverse events. Cardiothoracic Technique and Technologies VII, Session IA, Abstract 10.)

Most of these techniques assist OPCAB by decreasing the End Diastolic Volume (EDV) of the right ventricle, left ventricle, or both, with different rates of success. None of the techniques are designed to enhance coronary blood flow and perfusion pressure, which is believed to play a critical role in hemodynamic stability during OPCAB.

Assist Devices for Heart Failure

U.S. Pat. No. 6,387,037 B1 (Bolling et al.) provides a summary of assist devices that are known in the art. Both the existing LVADs and IABPe have considerable physiological limitations. First, the current forms of LVADs whether pulsatile or non-pulsatile, synchronized or unsynchronized, often result in an increase of intra-aortic pressure that persists even during subsequent cardiac systole. Second, the ideal IABP diastolic augmentation, with suprasystolic-diastolic pressure peak, can only be achieved in a small proportion of cases, due to technical issues or due to inadequate volume displacement. These limitations have largely been ignored, either due to a failure to recognize these problems, or due to lack of a practical solution. The prior art of assist devices and techniques always result in an increase in ventricular after-load and myocardial oxygen demand. These are serious limitations, which are detrimental to the heart muscle especially when the native heart is expected to make some contribution to circulation, e.g. during partial ventricular bypass, and during weaning from LVAD. However, diastolic volume augmentation by itself is not enough to alleviate this problem.

Our studies show that even with a counterpulsatile infusion of blood from the ventricles into the arterial system, peripheral resistance dictates that the pressure and volume dissipation follows an exponential decay over several cycles. The dissipation of volume and pressure is described by the formula:

y(t)=ce^{kt,}

where c is an arbitrary constant, k is a definite physical constant and t represents time.

Considering large volumes usually associated with LVAD function, the effects of a slow pressure and volume dissipation results in a marked elevation of afterload following an assisted beat. A physiological VAD capable of diastolic volume augmentation, and after-load reduction, is thus needed to provide a significant improvement in this vital aspect of cardiovascular technology. Thus, there is a need in the art for a more physiological cardiac assist device.

### SUMMARY OF THE INVENTION

It is an object of the present invention to provide a physiologically compatible cardiac assistance device and method.

As embodied and broadly described herein, the present invention provides a physiologically compatible cardiac assist device for cardiac assistance of the failing heart, and for use during coronary artery bypass surgery with or without cardiopulmonary bypass, said device comprising;

a first catheter for insertion into the left or the right atrium directly or via the great veins, or into the left ventricle of the patient;

a second catheter for insertion into the aorta directly, or via the femoral or brachial artery of the patient, said catheter also capable of carrying an intra-aortic balloon for diastolic counter-pulsation;

a pump for drawing blood through said first catheter and for delivering blood through said second catheter to the aorta, said pump is also capable of inflating a pneumatically driven intra-aortic balloon;

a control system for monitoring the performance of the patient's heart and for controlling operation of said pump to deliver blood and drive the intra-aortic balloon through said second catheter during only the diastolic phase of the cardiac cycle.

As embodied and broadly described herein, the present invention also provides a method of cardiac assistance to maintain blood circulation in the body of a patient during heart failure and coronary artery bypass surgery, said method comprising inserting a first catheter into the patient's venous system; inserting into the patient's arterial system a second catheter with an inflatable balloon attached near its distal end; positioning the distal end of said second catheter such that the balloon is located in the patient's descending thoracic aorta; monitoring the patient's heart with electrical sensors to determine the diastolic phase; and pumping blood from said patient through said first catheter and infusing said blood into said patient through said second catheter and simultaneously inflating the balloon such that the infusion of blood and the inflation of the balloon are both in synchrony with the patient's diastolic phase.

Another object of this invention is to address one of the major limitations of the existing ventricular assist devices, namely the ill-timed persistence of high after-load during ventricular assistance. The cardiac assist device of the present invention overcomes this defect. The device exploits the established principle of aortic diastolic counterpulsation and that of volume displacement to achieve a more physiologically compatible ventricular assist device (PCVAD). PCVAD is achieved by inserting a balloon in the aorta via the femoral artery, which is then inflated to displace blood during diastole. In addition the PCVAD is used to transfer blood to bypass diseased ventricle(s). The blood is withdrawn from the right or left side of the heart using and intra-vascular catheter, the blood is then pumped into the aorta via the catheter inserted into the femoral or the upper limb arteries. This allows off-loading of one or both ventricles, and allows direct augmentation of systemic pressures and flows, including those of the coronary arteries. Our animal studies shows that PCVAD offers significant reduction of both pre-load and after load, it improves ventricular function, and increases both aortic diastolic pressures and coronary flows. If the blood is withdrawn from the right side of the heart, the circuit can then be used as a pulsatile cardiopulmonary bypass machine with the insertion of an oxygenator.

One important advantage of this invention is the ability to apply both volume and pneumatic counterpulsation using a single intra-aortic catheter. Furthermore this catheter may be used both as inflow and an outflow catheter as described in U.S. Pat. No. 6,007,479 by Rottenberg et al (Dec. 28, 1999). The avoidance of multiple cannulation sites will minimize the risk of bleeding which is one of the major causes of death during of ventricular assistance.

PCVAD application can also prevent the side-effects usually associated with OPCAB procedures—i.e. significant deterioration of cardiac function due to impaired cardiac output, and poor coronary artery flow—by simultaneously reducing the cardiac pre-load and augmenting the diastolic pressure to increase the coronary flow and the mean arterial pressure. Unlike existing OPCAB pumps, the PCVAD does not require direct catheterization or suturing to the ascending aorta, it can be applied without opening the chest, thus allowing minimally invasive surgical procedures to be performed safely. Using an additional oxygenator, PCVAD can also be used as a conventional cardiopulmonary bypass pump with pulsatile flow.

Other advantages of using the PCVAD include the following;

(1) Enhanced coronary blood flow during OPCAB grafting:

(a) by rhythmic augmentation of the aortic root pressure during ventricular diastole at the time of maximum coronary blood flow; and

(b) by reducing the workload of the left and right ventricle by decreasing the End Diastolic Volume, and consequently the workload of the ventricle—this will reduce oxygen demand and consumption by the myocardium. The improved oxygenation of the myocardium results in more resistance to repetitive local ischemia.

(2) Enhanced systemic circulation, including cerebral and renal circulation.

(3) Reduces the need for administering high doses of ionotropic and vasoactive agents postoperatively in high-risk coronary patients who have an increased likelihood of developing low cardiac output following myocardial revascularization.

(4) It allows the OPCAB procedure to be used for higher risk cases including small and diffusely diseased vessels, patients with heart failure, haemodynamically unstable, left ventricular dysfunction, and those with significant cardiac enlargement.

(5) As the PCVAD has the advantage of using peripheral vessels to operate the device, this will increase the ability of performing minimally invasive CABG, and Endoscopic Robot-Assisted CABG.

The invention thus allows beating heart surgery to be performed for an increased range of indications. The PCVAD further enables anastomosis in more remote posterior areas under greater haemodynamic stability, and also protects the heart and other vital organs from warm ischaemia.

The device can also be used in various conditions that require cardiac assistance.

In summary, the operation of the PCVAD requires insertion of an inflow (to the pump) catheter into the left the right venous system, the insertion of an outflow (to the patient) cannula into the thoracic aorta directly or via the femoral or the brachial artery. The outflow cannula doubles up as an intra-aortic balloon pump. The blood is drained into a heparinized, primed pump tubing. Volume transfer and pneumatic pumping is synchronized with the diastolic phase of the cardiac cycle. Infusion volume and rate are controlled by adjusting the timing and the duration of pumping, according to the patient's need. However, the PCVAD should not be used for patients with significant aortic regurgitation, aortic aneurysm, or in cases of severe peripheral vascular diseases.

Other objects and features of the invention will become apparent by reference to the following description and the drawings.

### BRIEF DESCRIPTION OF THE DRAWINGS

The invention is further described, by way of example only, with reference to the accompanying drawings wherein

FIG. 1 is a schematic of the physiologically compatible cardiac assistance device of the present invention with the inflow cannula drawing blood from the left ventricle.

FIG. 2 is a schematic of the physiologically compatible cardiac assistance device of the present invention with the inflow cannula drawing blood from the right atrium.

FIG. 3 is a schematic of the physiologically compatible cardiac assistance device of the present invention with the inflow cannula drawing blood from the left atrium.

FIG. 4 is a schematic of the second catheter and the intra-aortic balloon.

In the drawings, preferred embodiments of the invention are illustrated by way of examples. It is to be expressly understood that the description and drawings are only for the purpose of illustration and are an aid for understanding. They are not intended to be a definition of the limits of the invention.

### DESCRIPTION OF THE PREFERRED EMBODIMENTS

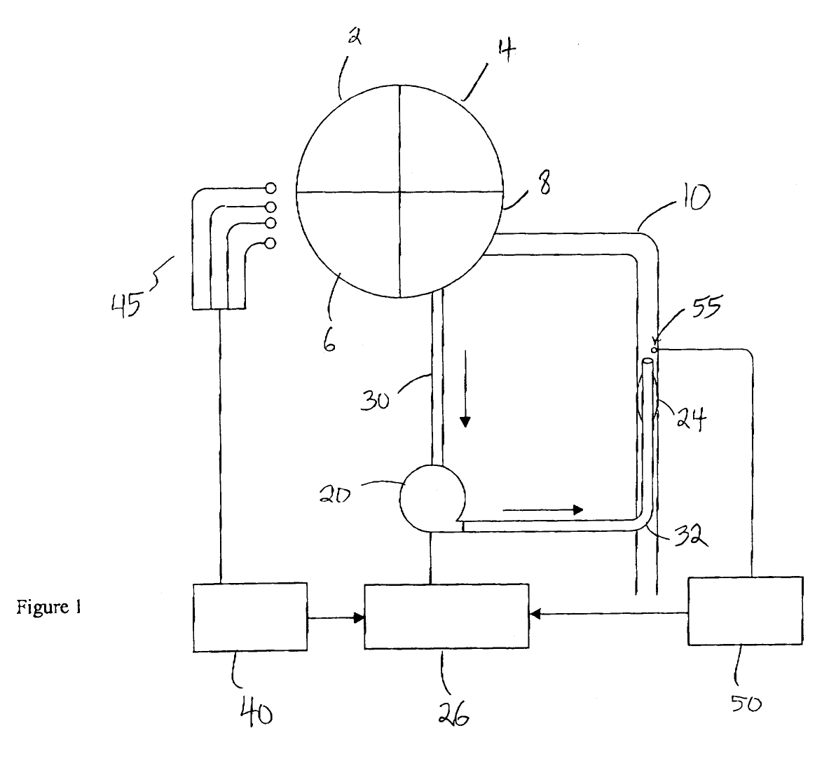

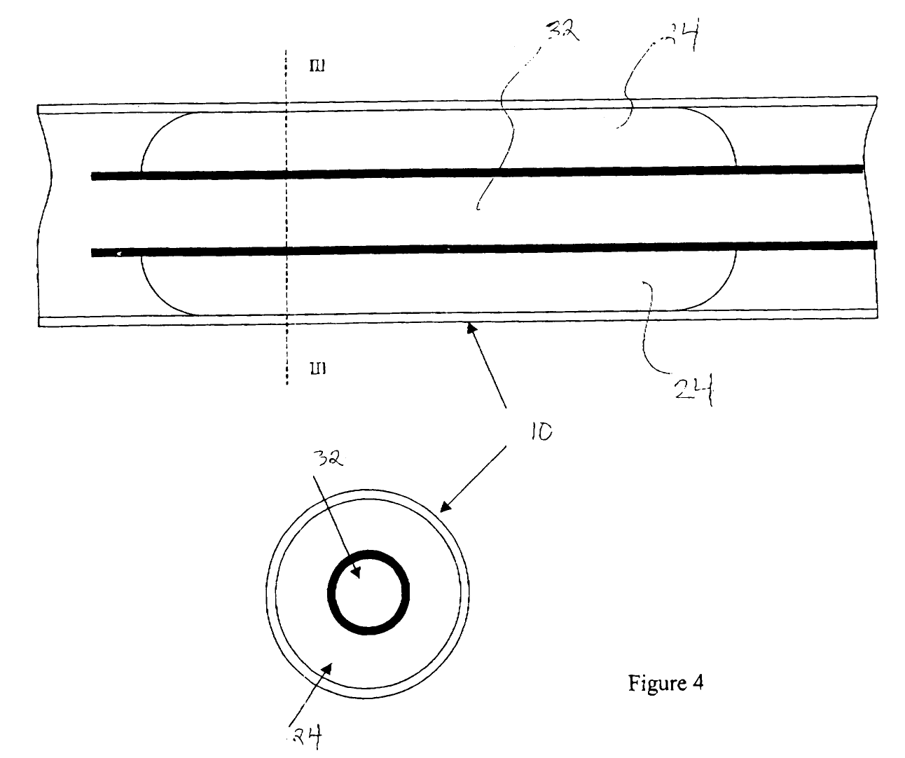

A physiologically compatible cardiac assistance device (also known as a physiologically compatible ventricular assistance device or PCVAD) connected to the circulatory system of a human patient. As depicted in FIG. 1, the PCVAD is used to assist a diseased or failing heart having a right atrium 2, a left atrium 4, a right ventricle 6, a left ventricle 8 and an aorta 10. The PCVAD has a pump 20 which draws blood through an inflow cannula 30 (a first catheter) connected to the left atrium or ventricle 8. The pump 20 impels the blood through a second catheter 32 into the aorta. The pump 20 also inflates a balloon 24 mounted near the tip of the second catheter 32. The intra-aortic balloon is mounted to the second catheter in the manner illustrated in FIG. 4.

A pump/balloon control system 26 receives electrical signals from an ECG monitor 40 and infuses blood and inflates the balloon 24 during diastole. The ECG monitor has four leads 45 connected to the patient to monitor the heart. The pump/balloon control system 26 also receives a signal from a pressure monitor 50 which in turn receives a signal from a pressure transducer 55 located in the aorta 10 upstream of the distal end of the second catheter 32. The pump/balloon control system 26 is thus able to regulate blood infusion into the aorta and inflation of the intra-aortic balloon to minimize afterload and preload. The device reduces preload by drawing blood from the first catheter. The device reduces afterload by confining the volume displacement within diastole and by the sudden deflation of the balloon at or before the end of diastole. Balloon deflation is triggered by the ECG R wave. Overall, the device assists circulation by volume infusion through the second catheter and by balloon inflation during diastole.

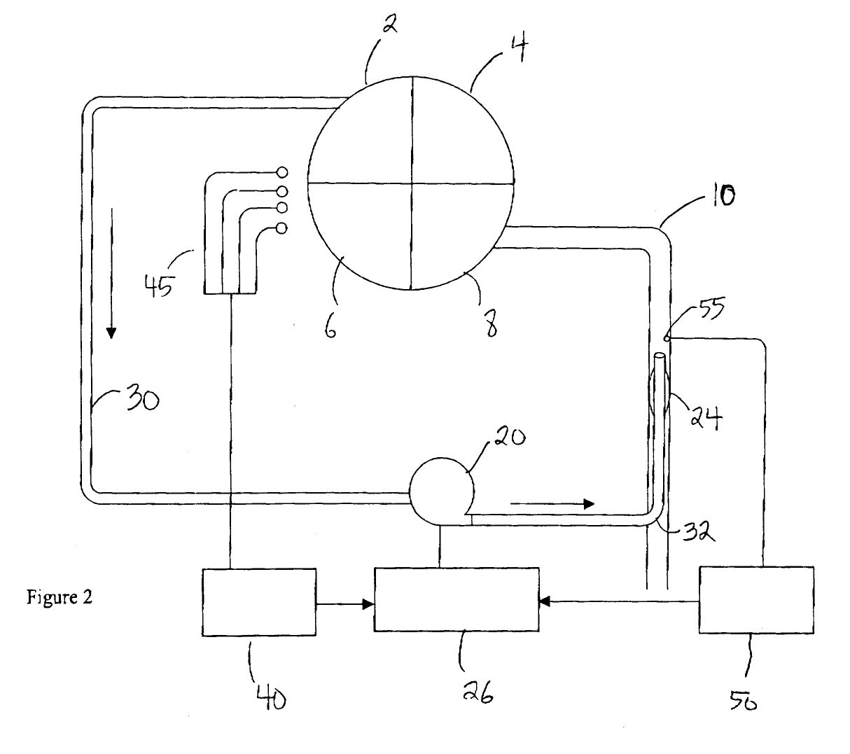

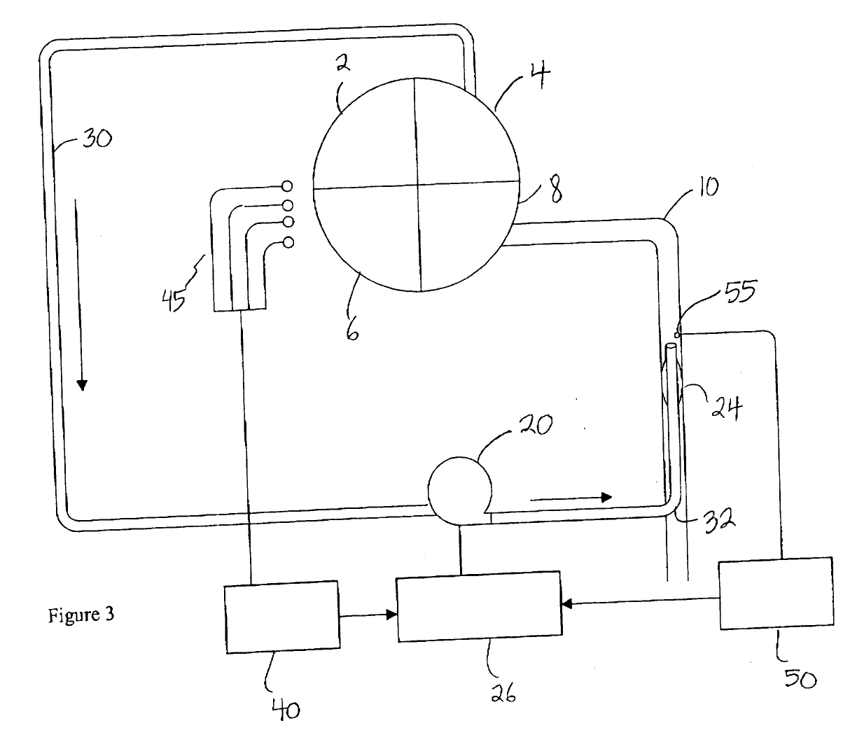

FIG. 2 depicts the first catheter 30 being connected to the right atrium 2 whereas FIG. 3 depicts the first catheter 30 being connected to the left atrium 4.

The PCVAD thus comprises two catheters, a first catheter 30 for inflow towards the pump 20 and a second catheter 32 for outflow away from the pump 20. The second (outflow) catheter 32 also has a second channel that leads to a pneumatically driven intra-aortic balloon 24 circumventing the lumen used for volume inflow. The pump 20 actively infuses the blood into the aorta at a rate of 1 to 5 liters/min, and triggers the inflation of the intra-aortic balloon 24.

The second catheter 32 (known as an arterial catheter) can be inserted via a femoral artery cut down and guided to a position in the descending or the abdominal aorta. The venous catheter can be inserted into the left or right atrium or the left ventricle directly or via the great veins.

The PCVAD is synchronized to eject blood and inflate the balloon during diastole. The pump will propel 20 ml to 50 ml of blood with each cardiac cycle. The pump can be de-activated during the rest of the cardiac cycle. A rhythmic mechanical blood pump aids the circulatory function of the heart that acts to provide internal counter pulsation. The pump will draw blood from the inflow cannula. The pump will propel between 1 to 5 litres/min of blood at a rate of between 50 to 100 beats per minute via the outflow cannula. The pump must be inactive during the rest of the cardiac cycle.

In the event where the heart is not beating or in the absence of a good ECG signal, the device can be switched manually to automatic mode whereby the pump continues to deliver blood to the rest of the body at an operator-determined heart rate and stroke volume.

The PCVAD set-up requires that the pump 20 be controlled by a control system 26, that comprises an ECG monitor and selection switches that allows for pressure wave recording and the adjustment of pump timing. The ECG monitoring is effected using electrodes that measure the electrical activity of the patient's heart and send an impulse to activate the cardiac assist device during the diastolic phase of the cardiac cycle. For safety reasons, a back-up battery source is provided to supplement the main power supply if the latter is unexpectedly cut off.

To achieve optimal effect, the blood pump needs to be correctly synchronized to the patient's cardiac cycle in real time. This is accomplished by depicting the patients ECG signals (R-waves and calculated Q-T interval) or the patient's arterial waveform such as the dicrotic-notch.

The tip of the arterial limb (outflow) catheter of the PCVAD is placed within the patient's thoracic aorta. The balloon pump component must be placed away from the renal arteries to avoid renal ischemia. Pumping of the blood starts with the onset of diastole and is closed down prior to the onset of systole.

During diastole the PCVAD pumps the blood to the aorta to be forced proximally into the coronary arteries and the main branches of the aortic arch, with increase of coronary and cerebral perfusion.

The purpose of doing so is twofold. Firstly, the increase in coronary perfusion pressure and myocardial oxygen supply leads to improvement of cardiac output, ejection fraction, and a decrease in heart rate. Secondly, by reducing Systemic venous return (in the case of right atrium-to-aorta configuration), right ventricular preload, pulmonary capillary wedge pressure, and left ventricular preload are reduced.

Transient differential (lower limb) cyanosis is expected while operating the PCVAD from the right atrium to the aorta. However, this side effect may be tolerated for transient assistance e.g. during OPCAB, or avoided by using an oxygenator.

The above description of preferred embodiments should not be interpreted in a limiting manner since other variations, modifications and refinements are possible within the spirit and scope of the present invention. The scope of the invention is defined in the appended claims and their equivalents.

What is claimed is:

1. A physiologically compatible cardiac assist device for cardiac assistance and use during coronary artery bypass surgery on a patient with or without cardiopulmonary bypass, said device comprising:
a first catheter for insertion into the left or the right atrium directly or via the great veins, or into the left ventricle of the patient;
a second catheter for insertion into the aorta via the femoral or brachial artery of the patient, said catheter also capable of carrying an intra-aortic balloon for diastolic counter-pulsation;
a pump for drawing blood through said first catheter and for delivering blood through said second catheter to the aorta, said pump capable of inflating a pneumatically driven intra-aortic balloon;
a control system for monitoring the performance of the patient's heart and for controlling operation of said pump to deliver blood and drive the intra-aortic balloon through said second catheter during only the diastolic phase of the cardiac cycle.

2. A cardiac assist device as claimed in claim 1 wherein said control system includes an electrocardiogram having electrodes that can be positioned to monitor the performance of the patient's heart to identify the cardiac cycle thereof and to determine the duration of the diastolic phase and the systolic phase thereof, said electrocardiogram being connected to deliver to said control system input signals representing the cardiac cycle of the patient's heart, said control system being effective to control operation of said pump in response to said input signals to ensure that said pump is actuated only during the diastolic phase of each cardiac cycle, operation of said pump being terminated before the onset of the systolic phase of each cardiac cycle.

3. A cardiac assist device as claimed in claims 1 or 2 including means for adjusting the duration and timing of actuation of the pump during each diastolic phase to control the volume of blood delivered through said second catheter.

4. A cardiac assist device as claimed in any one of claims 1 to 3 wherein said first catheter is sized to be inserted through an incision in the patient's venous system, said second catheter being sized for insertion through an incision in the patient's femoral artery and to be advanced through the femoral artery to a location at the patient's descending thoracic aorta.

5. A method of cardiac assistance to maintain blood circulation in the body of a patient during heart failure and coronary artery bypass surgery, said method comprising:
a) inserting a first catheter into the left atrium or the femoral or internal jugular vein of the patient;
b) inserting a second catheter into a femoral artery of the patient;
c) monitoring the performance of the patient's heart to identify the cardiac cycle thereof; and
d) drawing blood from said patient through said first catheter and returning blood to said patient through said second catheter in synchrony with the cardiac cycle of the patient such that blood is returned to the patient through said second catheter only during the diastolic phase of the cardiac cycle, flow of blood through said second catheter being terminated before the onset of the systolic phase.

6. The method of claim 5 wherein said first catheter is inserted to draw blood from the left atrium, superior vena cava or inferior vena cava.

7. The method of claims 5 or 6 wherein the second catheter is positioned to deliver blood into the descending thoracic aorta of the patient's blood circulatory system.

8. The method of any one of claims 5 to 7 wherein blood is delivered in a pressure pulse of duration and magnitude that is synchronized with the pumping action of the heart and that augments the diastolic pressure to increase coronary blood flow and mean arterial pressure.

9. The method of any one of claims 5 to 8 wherein blood is returned through the second catheter in a manner to replicate and augment the pumping action of the heart.

10. A method of cardiac assistance to maintain blood circulation in the body of a patient during heart failure and coronary artery bypass surgery, said method comprising:
a) inserting a first catheter into the patient's venous system;
b) inserting into the patient's arterial system a second catheter with an inflatable balloon attached near its distal end;
c) positioning the distal end of said second catheter such that the balloon is located in the patient's descending thoracic aorta;
d) monitoring the patient's heart with electrical sensors to determine the diastolic phase; and
e) pumping blood from said patient through said first catheter and infusing said blood into said patient through said second catheter and simultaneously inflating the balloon such that the infusion of blood and the inflation of the balloon are both in synchrony with the patient's diastolic phase.

11. The method of claim 10 wherein said first catheter is inserted into the left atrium or the femoral or internal jugular vein.

12. The method of claims 10 or 11 wherein the inflation of said balloon and the simultaneous infusion of blood into the aorta are controlled and synchronized by a control system.

13. The method of claim 12 wherein the control system receives input signals from electrocardiograph leads placed on the patient's body.
